(19) United States
(12) Patent Application Publication
(10) Pub. No.: US 2024/0358927 A1
(43) Pub. Date: Oct. 31, 2024

(54) DEVICES, SYSTEMS, AND METHODS FOR COMBINING AND/OR DELIVERING INJECTABLE MATERIALS

(71) Applicants: Boston Scientific Medical Device Limited, Galway (IE); Boston Scientific Scimed, Inc., Maple Grove, MN (US)

(72) Inventors: Nitesh Ghananil Baviskar, Kalyan West (IN); Benjamin Cleveland, Bellingham, MA (US); Sanjay Kumar Chandra, Pune (IN); Katie Knowles, Providence, RI (US); Junaid Mohammed Shaikh, Surat (IN); Marshall O'Hearn, Davenport, FL (US)

(73) Assignees: Boston Scientific Medical Device Limited, Galway (IE); Boston Scientific Scimed, Inc., Maple Grove, MN (US)

(21) Appl. No.: 18/647,377

(22) Filed: Apr. 26, 2024

Related U.S. Application Data
(60) Provisional application No. 63/462,154, filed on Apr. 26, 2023.

(51) Int. Cl. A61M 5/28; A61M 5/315; A61M 5/32

(52) U.S. Cl. CPC A61M 5/284; A61M 5/31511; A61M 5/3202

(57) ABSTRACT

Mixing systems for producing a mixture to deliver to a treatment site. An illustrative system may comprise a needle hub, a multi-reservoir system, and a syringe. The multi-reservoir system may comprise a plunger assembly including a first plunger and a second plunger, a barrel portion including a housing defining a first reservoir in fluid communication with a first port and configured to contain a first constituent and a second reservoir configured to contain a second constituent and in fluid communication with a second port, and a cap including a port and a lumen extending therethrough. A third constituent may be injected from the syringe into the first reservoir via the port of the cap to form a precursor. Actuating the plunger assembly relative to the barrel portion may cause the precursor and the second constituent to be delivered into the needle hub.

### CROSS-REFERENCE TO RELATED APPLICATIONS

This application claims the benefit of U.S. Provisional Patent Application Ser. No. 63/462,154 filed on Apr. 26, 2023, the disclosure of which is incorporated herein by reference.

### FIELD

The present disclosure relates generally to the field of devices for delivering injectable materials and/or compositions to a patient, and associated systems and methods. More particularly, the present disclosure relates to devices for combining constituents of injectable materials and/compositions, and associated systems and methods.

### BACKGROUND

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy. Various systems provide filler material to treatment sites to decrease the radiation dose to tissue surrounding radiation target sits (e.g., to shield the rectum during radiotherapy for prostate cancer). Such filler materials are often reactive, and therefore are generally combined/mixed immediately prior to or even during delivery to the patient.

Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily to increased procedure time and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with certain embodiments of the present disclosure, devices, systems, and methods for combining and/or delivering injectable materials are disclosed.

In a first example, a system for producing a mixture to deliver to a treatment site may comprise a needle hub, a multi-reservoir system, and a syringe defining a lumen configured to container a third constituent. The multi-reservoir system may comprise a plunger assembly including a first tubular plunger and a second tubular plunger, a barrel portion including a housing defining a first reservoir in fluid communication with a first port and configured to contain a first constituent and a second reservoir in fluid communication with a second port and configured to contain a second constituent and a first floating plunger movably disposed relative to the plunger assembly and at least partially within the first reservoir. A distal end of the syringe may be configured to be releasably coupled to the barrel portion such that actuation of the syringe causes the third constituent to be injected into the first reservoir via the lumen of the cap to mix with the first constituent and form a precursor. Actuating the plunger assembly relative to the barrel portion may cause the precursor and the second constituent to be delivered into the needle hub.

Alternatively or additionally to any of the examples above, in another example, the system may further comprise a cap removably coupled to a distal portion of the barrel portion, the cap including a port and a lumen extending from the port to a proximal end of the cap, wherein the distal end of the syringe is configured to be releasably coupled to the port of the cap.

In another example, a system for producing a mixture to deliver to a treatment site may comprise a needle hub, a multi-reservoir system, and a syringe defining a lumen configured to contain a third constituent. The multi-reservoir system may comprise a plunger assembly including a first tubular plunger and a second tubular plunger, a barrel portion including a housing defining a first reservoir in fluid communication with a first port and configured to contain a first constituent and a second reservoir in fluid communication with a second port and configured to contain a second constituent, a first floating plunger disposed at least partially within a lumen of the first tubular plunger and at least partially within the first reservoir, and a cap removably coupled to a distal portion of the barrel portion, the cap including a port and a lumen extending from the port to a proximal end of the cap. A distal end of the syringe may be configured to be releasably coupled to the port of the cap such that actuation of the syringe causes the third constituent to be injected into the first reservoir via the lumen of the cap to mix with the first constituent and form a precursor. Actuating the plunger assembly relative to the barrel portion may cause the precursor and the second constituent to be delivered into the needle hub.

Alternatively or additionally to any of the examples above, in another example, the system may further comprise a removable retainer positioned adjacent to a proximal end of the barrel portion, the removable retainer limiting movement of the plunger assembly relative to the barrel portion.

Alternatively or additionally to any of the examples above, in another example, the system may further comprise a removable retainer positioned between a proximal end of the barrel portion and a proximal end of the plunger assembly, the removable retainer limiting movement of the plunger assembly relative to the barrel portion.

Alternatively or additionally to any of the examples above, in another example, the system may further comprise a plug removably coupled with the port of the cap.

Alternatively or additionally to any of the examples above, in another example, the cap may further comprise a cavity in selective fluid communication with the second reservoir.

Alternatively or additionally to any of the examples above, in another example, the system may further comprise a floating seal disposed within the cavity of the cap, the floating seal configured to selectively seal the second port.

Alternatively or additionally to any of the examples above, in another example, the first floating plunger may be unsecured to the plunger assembly or the barrel portion.

Alternatively or additionally to any of the examples above, in another example, the system may further comprise a second floating plunger disposed at least partially within a lumen of the second tubular plunger and at least partially within the second reservoir.

Alternatively or additionally to any of the examples above, in another example, the system may further comprise a second floating plunger movably disposed relative to the plunger assembly and at least partially within the second reservoir.

Alternatively or additionally to any of the examples above, in another example, the second floating plunger may be unsecured to the plunger assembly or the barrel portion.

Alternatively or additionally to any of the examples above, in another example, the system may further comprise a needle that is configured to be coupled to the needle hub.

Alternatively or additionally to any of the examples above, in another example, the needle hub may comprise a first lumen in fluid communication with the first reservoir of the barrel portion via the first port, a second lumen in fluid communication with the second reservoir of the barrel portion via the second port, a central lumen configured to be in fluid communication with a needle, and a mixing region connecting the first and second lumens with the central lumen.

Alternatively or additionally to any of the examples above, in another example, the needle hub may be removably coupled to a distal end region of the barrel portion of the multi-reservoir system.

Alternatively or additionally to any of the examples above, in another example, the plunger assembly and the barrel portion may be assembled in a telescoping arrangement.

In another example, a kit for producing a mixture for delivery to a treatment site may comprise a multi-reservoir system, a syringe defining a lumen configured to contain a third constituent, and an injection system. The multi-reservoir system may comprise a plunger assembly including a first tubular plunger and a second tubular plunger, a barrel portion including a housing defining a first reservoir in fluid communication with a first port and configured to contain a first constituent and a second reservoir in fluid communication with a second port and configured to contain a second constituent, a first floating plunger disposed at least partially within a lumen of the first tubular plunger and at least partially within the first reservoir, and a cap removably coupled to a distal portion of the barrel portion, the cap including a port and a lumen extending from the port to a proximal end of the cap. The injection system may comprise a needle hub and a needle coupled to the needle hub.

In another example, a kit for producing a mixture for delivery to a treatment site may comprise a multi-reservoir system, a syringe defining a lumen configured to contain a third constituent, and an injection system. The multi-reservoir system may comprise a plunger assembly including a first tubular plunger and a second tubular plunger, a barrel portion including a housing defining a first reservoir in fluid communication with a first port and configured to contain a first constituent and a second reservoir in fluid communication with a second port and configured to contain a second constituent, a first floating plunger movably disposed relative to the plunger assembly and at least partially within the first reservoir, and a cap removably coupled to a distal portion of the barrel portion, the cap including a port and a lumen extending from the port to a proximal end of the cap. The injection system may comprise a needle hub and a needle coupled to the needle hub.

Alternatively or additionally to any of the examples above, in another example, the kit may further comprise a removable retainer positioned between a proximal end of the barrel portion and a proximal end of the plunger assembly, the removable retainer configured to limit movement of the plunger assembly relative to the barrel portion.

Alternatively or additionally to any of the examples above, in another example, the kit may further comprise a removable retainer positioned adjacent to a proximal end of the barrel portion, the removable retainer configured to limit movement of the plunger assembly relative to the barrel portion.

Alternatively or additionally to any of the examples above, in another example, the kit may further comprise a plug removably coupled with the port of the cap.

Alternatively or additionally to any of the examples above, in another example, the kit may further comprise a second floating plunger disposed at least partially within a lumen of the second tubular plunger and at least partially within the second reservoir.

Alternatively or additionally to any of the examples above, in another example, the kit may further comprise a second floating plunger movably disposed relative to the plunger assembly and at least partially within the second reservoir

Alternatively or additionally to any of the examples above, in another example, the kit may further comprise a connector, the connector configured to couple the needle hub to an additional syringe.

In another example, a method for producing a mixture with a mixing system to deliver to a treatment site may comprise coupling a distal end of a syringe with a first port of the multi-reservoir system, injecting a first constituent into a first reservoir of the multi-reservoir system to mix the first constituent with a second constituent contained in the first reservoir of the multi-reservoir system and form a precursor, coupling a needle hub having a mixing region to a distal end of the multi-reservoir system, and actuating a plunger assembly to distally advance the precursor and a third constituent disposed within a second reservoir of the multi-reservoir system into the mixing region of the needle hub to form an injectable mixture.

Alternatively or additionally to any of the examples above, in another example, the method may further comprise removing a retainer from the plunger assembly prior to actuating the plunger assembly. Alternatively or additionally to any of the examples above, in another example, the method may further comprise actuating the plunger assembly after removing a plug from a port of a cap of the multi-reservoir system and prior to removing the cap to purge air from the multi-reservoir system.

Alternatively or additionally to any of the examples above, in another example, the method may further comprise removing a cap from a distal end region of the multi-reservoir system prior to coupling the needle hub to the distal end region of the multi-reservoir system.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure. Moreover, reference characters may indicate elements in some figures which are illustrated in other figures, and which, for the sake of brevity, are described only with reference to the other figures.

The detailed description will be better understood in conjunction with the accompanying drawings, wherein like reference characters represent like elements, as follows:

FIG. 1 depicts an exploded perspective view of an illustrative mixing system for mixing an injectable material;

FIG. 2A depicts a cross-sectional view of the multi-reservoir system of the mixing system of FIG. 1, taken at line 2A-2A;

FIG. 2B depicts a cross-sectional view of the multi-reservoir system of the mixing system of FIG. 1, taken at line 2B-2B;

FIG. 3 depicts a side view of the illustrative multi-reservoir system with the plug removed;

FIG. 4 depicts a side view of the multi-reservoir system coupled with the syringe;

FIG. 5 which depicts a side view of the multi-reservoir system coupled with the syringe in a dispensed configuration;

FIG. 6 depicts a perspective view of the illustrative multi-reservoir system and syringe being shaken;

FIG. 7 depicts a side view of the illustrative multi-reservoir system with the air/excess fluid purged;

FIG. 8 depicts a side view of the illustrative multi-reservoir system with the cap and syringe removed;

FIG. 9 depicts a perspective view of the illustrative multi-reservoir system with the retainer removed;

FIG. 10A depicts a perspective view of the unassembled illustrative injection system and saline syringe;

FIG. 10B depicts a perspective view of an assembled illustrative injection system and saline syringe;

FIG. 10C depicts a perspective view of the unassembled illustrative injection system and saline syringe;

FIG. 11 depicts a side view of the unassembled injection system and multi-reservoir system;

FIG. 12 depicts a side view of the assembled injection system and multi-reservoir system;

FIG. 13 depicts the cross-sectional view of the assembled injection system and multi-reservoir system taken at line 13-13 of FIG. 12;

FIG. 14 depicts the cross-sectional view of FIG. 13 in a dispensed configuration; and

FIG. 15 which depicts an alternative cross-sectional view of the assembled injection system and multi-reservoir system in a dispensed configuration taken at line 15-15 of FIG. 14.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to placing an injectable material between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). Certain aspects of the present disclosure include displacing and/or shielding a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injected as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, constituents of an injectable material are combined by a system formed in accordance with various principles of the present disclosure and injected into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally occurring and synthetic hydrogel forming polymers, polymer mixtures and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more constituents of an injectable material are provided separately, and are combined by a device, system, and method in accordance with various principles of the present disclosure to form an injectable material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more constituents combined by a device, system, or method formed in accordance with various principles of the present disclosure. In accordance with various principles of the present disclosure, a composition to be injected into a patient may be a combination of two or more constituents combined by a device, system, or method such as formed in accordance with various principles of the present disclosure. For instance, devices, systems, or methods of the present disclosure may be used to combine a first constituent and a second constituent for injection into a patient. The first constituent may be a precursor, e.g., a first constituent to be combined with an additional constituent to form the injectable compound. The second constituent may be an accelerator, an activating agent, a catalyst, an initiator, etc., which, upon combination with the precursor, produces the injectable compound, such as by altering the chemical composition or structure of the first constituent or precursor. The constituents may be combined prior (e.g., immediately prior) to delivery (e.g., injection) or as the constituents are being delivered to the patient so that the injectable material does not have time to form into a structure which may be difficult to inject or otherwise deliver to the patient. As such, the combination of the first constituent or precursor and the second constituent may be such that the injectable compound attains its desired properties and/or reaches its final form in situ.

In some embodiments, the injectable material is formed of a first constituent, a second constituent, and a third constituent. For instance, for various reasons it may be desirable to provide a first, precursor constituent in a solid form (e.g., to be more stable for storage and/or transport). The first constituent is combinable with the third constituent, and the thus-formed combined composition (which may be referenced as the precursor) is then combinable with the second constituent once the medical professional is ready to deliver (e.g., inject) the injectable material to the patient. The second constituent may facilitate a crosslinking interaction between the first and third constituents, for example, by initiating or accelerating the crosslinking interaction of the first and third constituents. Typically, one or more of the constituents of the injectable materials are biocompatible polymers. In some aspects, one of the first, second, or third constituents is a reactive polymer, such as a cross-linkable and/or hydrophilic polymer constituent (e.g., polyethylene glycol (PEG)), and one of the first, second, third constituent is a diluent (e.g., mostly water) in which a solid or semi-solid form of the reactive polymer is dissolved or dispersed, and/or with which the reactive polymer is cross-linked (or at least cross-linkable, such as upon further combination with the second constituent), to form a precursor. Another of the first, second, or third constituents may be an accelerator, an accelerant, an activating agent, a catalyst, an initiator, etc. (such terms being used interchangeably herein without intent to limit), combinable and reactive with the precursor to form the desired injectable material. In one example of an embodiment, a first constituent, in the form of a reactive polymer (specifically, PEG) that has been derivatized with reactive electrophilic groups (specifically, succinimide ester groups), is mixed with a third constituent, in the form of a cross-linking agent (specifically, trilysine, which contains multiple nucleophilic groups, specifically, amino groups), under acidic pH conditions where the succinimide ester groups and the amino groups do not react to any significant degree. When this mixture is combined with a second constituent, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel. It will be appreciated that reference to “first”, “second”, or “third” is not intended to connote a particular nature of the material or the order in which the material is combined. As such, “first”, “second”, and “third” may be used to reference any of three constituents forming an injectable material in accordance with various principles of the present disclosure. A non-limiting example of such constituents combinable by devices, systems, or methods in accordance with various principles of the present disclosure includes a reactive constituent, a diluent with which the reactive constituent is to be combined to form a precursor, and an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, the first constituent is a biocompatible polymeric constituent. More particularly, in one example of an embodiment, the first constituent is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris(hydroxymethyl) methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa-s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

In some embodiments, a multi-reservoir system includes separate reservoirs for constituents to be combined to form the injectable material to be delivered to the patient by an injection system. In some embodiments, a first constituent and a second constituent are separately contained within a first reservoir and a second reservoir, respectively, of a multi-reservoir device. A third constituent may be contained in a separate reservoir defining a third reservoir of the multi-reservoir system. To deliver the injectable material, the constituents of the first and third reservoirs are combined within the first reservoir (e.g., to form a precursor) and then the constituents of the first and second reservoirs are injected together into the patient. The multi-reservoir device may or may not mix the contents of the first reservoir with the contents of the second reservoir. For instance, the multi-reservoir device may deliver and inject the constituents of the injectable material to an injection system, with the injection system including a mixer component configured to mix the constituents from the first reservoir of the multi-reservoir device with the contents from the second reservoir of the multi-reservoir device as those contents are injected from the multi-reservoir device and the injection system into the patient. The already-combined first and third constituents are combined with the second constituent to form the desired form, structure, composition, properties, etc., of the injectable material to be delivered and deposited within the patient. The final form, structure, composition, properties, etc., of the injectable material may be attained once the combined constituents are within the patient.

The present disclosure provides devices, systems, and methods for combining constituents to form an injectable composition, and corresponding medical devices, systems, and methods for use thereof and/or delivery to a treatment site of a patient. According to some aspects of the present disclosure, such as described above, a multi-reservoir system may include a plurality of reservoirs for the one or more constituents of the injectable material and for combinations of such constituents. It will be appreciated that terms such as chamber, reservoir, container, vial, lumen, etc., may be used interchangeably herein without intent to limit, to refer to elements which contain, convey, hold, transport, collect, etc., a constituent (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for the reservoir of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more constituents to form an injectable material may include and/or be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes. According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 pound-force (lb·f) to about 25 lb·f, such as from about 10 lb·f to about 20 lb·f, e.g., about 15 lb·f. The loads measured for a given gel concentration may vary for different needles and flow rates.

According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein.

According to some aspects of the present disclosure, a combining device or system can be included in a kit for introducing an injectable material into a patient, whereby the injectable material can include any of a variety of suitable compositions. Kits or systems may be configured to store one or more of the constituents of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain constituents that have sterility, purity, and preparation that is pharmaceutically acceptable. Solvents/solutions may be provided in the kit or separately. The kit may include one or more syringes and/or needles for mixing and/or delivery of the injectable material, and/or for additional aspects of the procedure in which the injectable material is to be used. The kit or system may comprise various constituents as set forth herein. For instance, a target site into which an injectable material is to be delivered may be pre-treated using one or more constituents of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of constituents of an injectable material. In some aspects, the injectable material is a combination of a first constituent, a second constituent, and a third constituent, such as described above. A combining and/or delivery system formed in accordance with various principles of the present disclosure facilitates combining/mixing of the various constituents of an injectable material. Additionally or alternatively, a combining and/or delivery system formed in accordance with various principles of the present disclosure facilitates delivery of the injectable material to an injection system configured to deliver (e.g., inject) and/or deposit the injectable material into a patient (e.g., to a target site within the patient). More particularly, various aspects of the present disclosure simplify assembly, alignment, mixing, dispensing, etc., of an injectable material which is combined or blended or mixed from separate constituents before delivery to a patient.

In some aspects, a combining and/or delivery system formed in accordance with various principles of the present disclosure includes two or more distinct lumens, chambers, or reservoirs for respective first and second constituents of an injectable material to be delivered and injected into a patient. A third constituent may be included in a separate device. The present disclosure facilitates combination of the separately-provided first and third constituents before a procedure, as well as combination of the recently-combined first and third constituents with the second constituent for injection into the patient. In particular, an example of an embodiment of a mixing and/or delivery system disclosed herein includes a combining system configured to facilitate the combining of the first, second, and third constituents of an injectable material before delivery to a patient. As described above, it may be desirable to provide the first, second, and third constituents separate from one another for combination only once a procedure is to be performed utilizing the injectable material formed by combining the first, second, and third constituents.

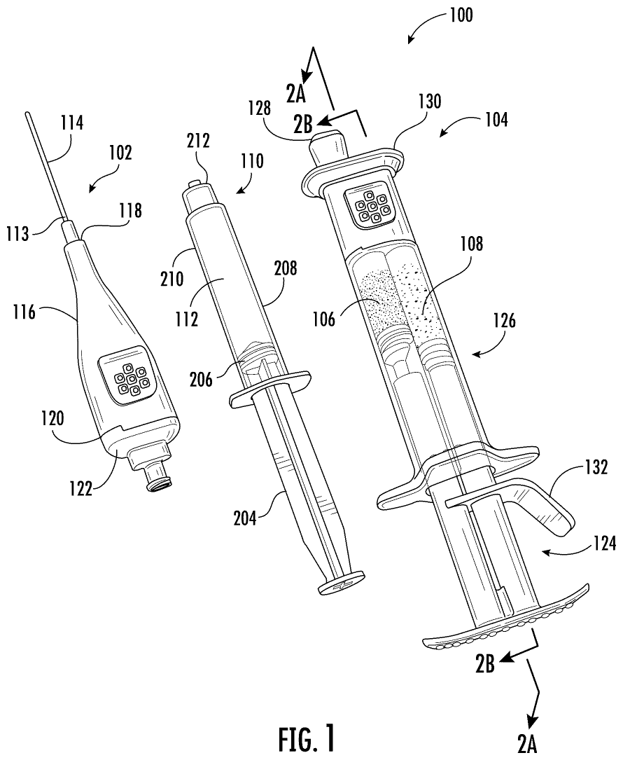

Turning to the drawings, FIG. 1 depicts an exploded perspective view of an illustrative combining and/or delivery system 100 in accordance with certain aspects of the present disclosure for mixing an injectable material. In some embodiments, the combining and/or delivery system 100 may be provided as a kit which may include, but is not limited to, a needle assembly or injection system 102 releasably attachable to a multi-reservoir system 104, a first constituent 106 disposed within a chamber or lumen of the multi-reservoir system 104, a second constituent 108 disposed within another chamber or lumen of the multi-reservoir system 104, a syringe 110, such as, but not limited to a diluent syringe, and a third constituent 112 disposed within a chamber or lumen of the syringe 110. The multi-reservoir system 104 may be used to transport the first and second constituents 106, 108 and the syringe 110 may be used to transport the third constituent 112 of an injectable material to a facility and/or location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. As described above, the first, second, and third constituents 106, 108, 112 may be mixed or combined at the time of treatment to form an injectable material.

The injection system 102 may include a needle 114 which can be any needle of this disclosure suitable for hydrodissection as well as delivering the injectable material (e.g., a gel composition) to the treatment site. A proximal end 113 of the needle 114 can be connected to a distal end 118 of a needle hub 116 (e.g., the needle 114 can be overmolded to connect to the needle hub 116). The proximal end 120 of the needle hub 116 may be attached to a distal end of a connector 122. In some embodiments, it is contemplated that the needle 114 may be replaced with a catheter tube or similar structure to reach a target location deeper in the body.

The syringe 110 may include a plunger 204, a plunger seal 206, a barrel 208 defining a lumen 210 extending therethrough, and a Luer lock 212 adjacent to a distal end of the barrel 208. While not explicitly shown, in some embodiments, the syringe 110 may further include a cap removably disposed over the Luer lock 212. The plunger 204 and plunger seal 206 may be axially and/or longitudinally slidable with respect to the barrel 208 to push materials out of and/or to aspirate materials into the lumen 210 of the barrel 208. When so provided, the cap may provide a fluid-tight seal with a distal opening of the barrel 208 to allow fluid, such as the third constituent, or diluent 112 to be stored or pre-loaded within the lumen 210.

The multi-reservoir system 104 of the overall system 100 may generally include a plunger assembly 124, a barrel portion 126, a first plug or cap 128, a second plug or cap 130, and a retainer 132. Briefly, a distal portion of the plunger assembly 124 may be slidably disposed within a proximal portion of the barrel portion 126. The barrel portion 126 may be configured to receive a third constituent 112 from the syringe 110 to first mix two constituents 106, 112 to form a precursor and then to mix the precursor with an accelerator 108 to form an injectable composition. In some cases, at least some of the mixing may occur within the multi-reservoir system 104 and some of the mixing may occur within the injection system 102, as will be described in more detail herein.

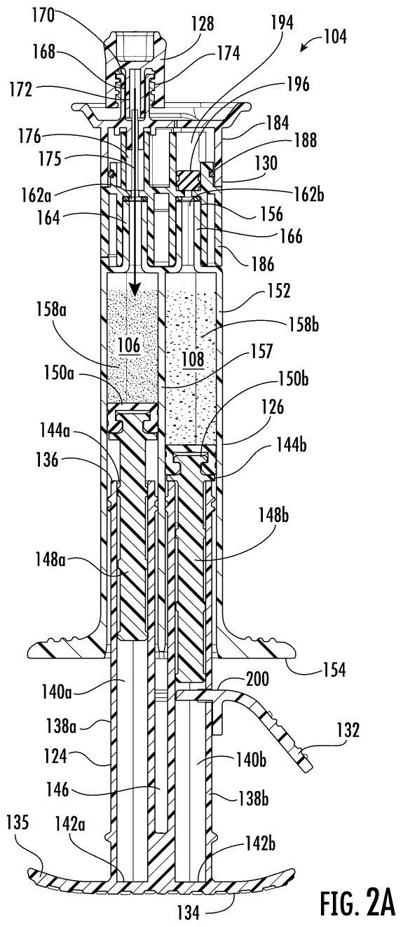

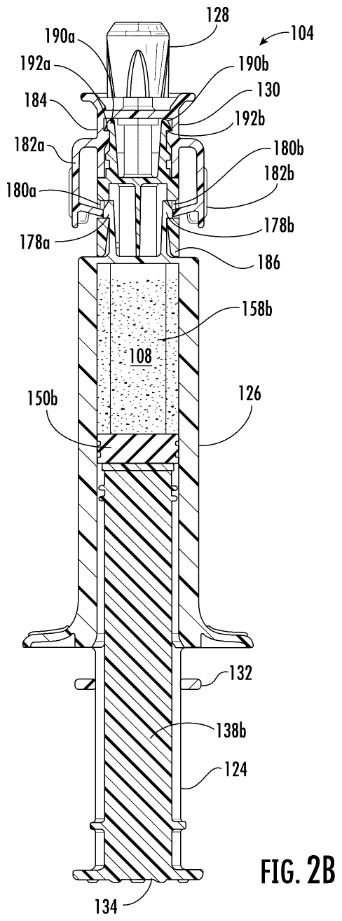

Referring additionally to FIG. 2A, which depicts a cross-sectional view of the multi-reservoir system 104 taken at line 2A-2A of FIG. 1 and FIG. 2B which depicts a cross-sectional view of the multi-reservoir system 104 taken at line 2B-2B of FIG. 1, the plunger assembly 124 may extend from a generally planar proximal end 134 to a distal end 136. The proximal end 134 may define a flange or an actuation member 135 for depressing or actuating the plunger assembly 124. In some cases, a surface of the actuation member 135 may include texturing such as, but not limited to, raised bumps, ridges, etc. to improve the gripability of the proximal end 134. A first plunger 138a and a second plunger 138b may extend distally from the actuation member 135 towards the distal end. The first plunger 138a may be generally tubular and may define a lumen or cavity 140a extending distally from a closed proximal end 142a to a distal opening 144a. Similarly, the second plunger 138b may be generally tubular and may define a lumen or cavity 140b extending distally from a closed proximal end 142b to a distal opening 144b. The first and second plungers 138a, 138b may be laterally spaced and arranged to extend parallel to one another in side-by-side arrangement. In some examples, there may be a gap or spacing 146 between the first and second plungers 138a, 138b to allow the plunger assembly 124 to be axially displaced within the barrel portion 126. The gap 146 may be sized to allow the plunger assembly 124 to actuate or advance distally relative to a dividing wall 157 of the barrel portion 126. The first and second plungers 138a, 138b may have a generally semi-circular or “D” shaped outer shape along at least some of the length thereof. Similarly, the lumens 140a, 140b may also have a generally semi-circular or “D” shaped cross-sectional shape. However, this is not required. Other shapes may be used as desired.

A first floating plunger 148a may be movably disposed relative to the plunger assembly 124 and/or the barrel portion 126. In some embodiments, the first floating plunger 148a may be movably disposed within at least a portion of the first lumen 140a. The first floating plunger 148a may float within the first lumen 140a. For example, the first floating plunger 148a may not be secured to either the plunger assembly 124 or the barrel portion 126. A distal end of the first floating plunger 148a may extend into a first reservoir 158a of the barrel portion 126. The first floating plunger 148a may include a stopper or seal 150a positioned adjacent the distal end thereof. The seal 150a may be sized and shaped to provide a fluid tight seal between the first floating plunger 148a and a wall of the first reservoir 158a of the barrel portion 126.

A second floating plunger 148b may be movably disposed relative to the plunger assembly 124 and/or the barrel portion 126. In some embodiments, the second floating plunger 148b may be movably disposed within at least a portion of the second lumen 140b. The second floating plunger 148b may float within the second lumen 140b. For example, the second floating plunger 148b may not be secured to either the plunger assembly 124 or the barrel portion 126. A distal end of the second floating plunger 148b may extend into a second reservoir 158b of the barrel portion 126. The second floating plunger 148b may include a stopper or seal 150b positioned adjacent the distal end thereof. The seal 150b may be sized and shaped to provide a fluid tight seal between the second floating plunger 148b and a wall of the second reservoir 158b of the barrel portion 126.

It is contemplated that in some embodiments, the plunger assembly 124 may have a non-tubular structure configured to engage a lumen (not explicitly shown), if so provided, of the first and/or second floating plungers 148a, 148b. For example, in some embodiments, the first and/or second plungers 138a, 138b may have a generally solid cross-section along at least a portion of the length thereof.

In some embodiments, the plunger assembly 124 may be formed as a single monolithic structure. In other embodiments, the plunger assembly 124 may be formed as two or more distinct components that are subsequently coupled together. In some cases, the actuation member 135 and the first and second plungers 138a, 138b may be formed as a single monolithic structure and the first and second floating plunger 148a, 148b and the seals 150a, 150b may be provided as separate components. This is just one example. Other configurations may be used, as desired.

The barrel portion 126 may include a body portion 152 extending from a proximal end 154 to a distal end 156. In some examples, the proximal end 154 may include a flange configured to provide a gripping region to allow the user to actuate or advance the plunger assembly 124 relative to the barrel portion 126. The body portion 152 may define a first chamber, lumen, or reservoir 158a and a second chamber, lumen, or reservoir 158b. The first and second reservoirs 158a, 158b may be laterally spaced and arranged to extend parallel to one another in side-by-side arrangement. The first and second reservoirs 158a, 158b may be fluidly isolated by a dividing wall 157. In some embodiments, the first and second reservoirs 158a, 158b may also have a generally semi-circular or “D” shaped cross-sectional shape configured to receive the first and second plungers 138a, 138b and the first and second floating plungers 148a, 148b. However, this is not required. Other shapes may be used as desired. In some embodiments, the barrel portion 126 may be formed as a single monolithic structure. In other embodiments, the barrel portion 126 may be formed as two or more distinct components that are subsequently coupled together.

In some examples, the first and second reservoirs 158a, 158b may reduce in cross-sectional dimension towards the distal end 156 of the barrel portion 126 to form a first port 164 and a second port 166. The first port 164 may be in fluid communication with the first reservoir 158a and a distal opening 162a. The second port 166 may be in fluid communication with the second reservoir 158b and a distal opening 162b. The first and second ports 164, 166 may be configured to be releasably coupled to the injection system 102, as will be described in more detail herein.

The first and second reservoirs 158a, 158b may be sized and shaped to receive at least a distal portion of the plunger assembly 124 which may be slidably disposed within the first and second reservoirs 158a, 158b of the barrel portion 126. For example, a distal end region of the first plunger 138a and a distal portion of the first floating plunger 148a may be disposed within the first reservoir 158a while a distal portion of the second plunger 138b and a distal portion of the second floating plunger 148b may be disposed within the second reservoir 158b.

In some embodiments, the cross-sectional dimension and/or shape of the first and second reservoirs 158a, 158b may decrease from the proximal end 154 to the distal end 156 of the barrel portion 126, although this is not required. The cross-sectional dimension may decrease in an abrupt step-wise manner to form discrete transitions in the cross-sectional dimension or the cross-sectional dimension may gradually taper.

The reservoirs 158a, 158b may be sized to hold a desired volume of the respective first or second constituent 106, 108. For example, the first constituent 106 may be held in the first reservoir 158a and the second constituent 108 may be held in the second reservoir 158b. The first floating plunger 148a and seal 150a may form a fluid tight seal at a proximal end region of the first reservoir 158a to allow a fluid to be stored in the first reservoir 158a while the plug 128 and cap 130 may selectively fluidly seal the distal opening 162a. The second floating plunger 148b and seal 150b may form a fluid tight seal at a proximal end region of the second reservoir 158b to allow a fluid to be stored in the second reservoir 158b while and cap 130 may selectively fluidly seal the distal opening 162b. In some examples, the volume of at least one of the reservoirs 158a, 158b may be increased or decreased as the constituents 106, 108 are moved within the multi-reservoir system 104.

A plug 128 may be releasably coupled to a port 168 of the cap 130. The plug 128 may be sized and shaped to be disposed over and fluidly seal an opening 170 of the port of the cap 130. In some examples, the plug 128 may include an inner region 172 that extending into the port 168 of the cap 130 and an outer region 174 that is surrounds an outer surface the port 168 of the cap 130. While not explicitly shown, the plug 128 may include an elastomeric or deformable sealing material disposed on an interior surface thereof and configured to contact the distal end of the port 168 of the cap 130 to form a fluid tight seal between the plug 128 and port 168 of the cap 130. The plug 128 may threadably engage the port 168 of the cap 130. However, other coupling mechanisms may be used, as desired, such as, but not limited to, friction fits, snap fits engagements, rotational locks, etc. In some examples, the plug 128 may be replaced with a valve or another flow control mechanism configured to selectively fluidly seal the opening 170.

A cap 130 may be releasably coupled to the distal end 156 of the barrel portion 126. The cap 130 may be sized and shaped to be disposed over and fluidly seal the second distal opening 162b of the barrel portion 126. When the plug 128 is assembled with the cap 130, the plug 128 and cap 130 may fluidly seal the first distal opening 162a of the barrel portion 126. When the plug 128 is uncoupled from the cap 130, a lumen 176 extending from the distal end of the port 168 to a proximal end of the cap 130 provides a fluid path 175 from the port 168 of the cap 130 to the first reservoir 158a. While not explicitly shown, the cap 130 may include an elastomeric or deformable sealing material disposed on an interior surface thereof and configured to contact the distal end 156 of the barrel portion 126 to form a fluid tight seal between the cap 130 and second port 166 of the barrel portion 126. The cap 130 may form a snap fit with the distal end 156 of the barrel portion 126. However, other coupling mechanisms may be used, as desired, such as, but not limited to, friction fits, threaded engagements, rotational locks, etc. In some examples, the cap 130 may be replaced with a valve or another flow control mechanism configured to selectively fluidly seal the distal opening 162b.

For example, when the cap 130 is assembled with the distal end region of the barrel portion 126, one or more tabs 178a, 178b of the barrel portion 126 may be received within one or more mating apertures 180a, 180b of the cap 130. The one or more apertures 180a, 180b may be disposed below one or more buttons 182a, 182b of the cap 130 such that actuating (e.g., depressing) the one or more buttons 182a, 182b is configured to disengage the one or more tabs 178a, 178b of the barrel portion 126 from the one or more mating apertures 180a, 180b to allow the cap 130 to be uncoupled from the multi-reservoir system 104. However, other coupling mechanisms may be used, as desired, such as, but not limited to, friction fits, threaded engagements, rotational locks, etc.

The cap 130 may include a first portion 184 and a second portion 186 with an O-ring 188 or other sealing member disposed therebetween. The second portion 186 may include one or more latches 190a, 190b configured to engage one or more mating ledges or protrusions 192a, 192b of the first portion 184. In some cases, the first portion 184 may include the one or more buttons 182a, 182b configured to releasably secure the cap 130 to the barrel portion 126 while the second portion 186 may include a chamber 194 configured to receive fluid from the second reservoir 158b during an air purge, as will be described in more detail herein. The chamber 194 may include a floating seal 196 configured to selectively seal the distal opening 162b. For example, the floating seal 196 may be configured to move away from the distal opening 162b in response to pressure generated through actuation of the plunger assembly 124.

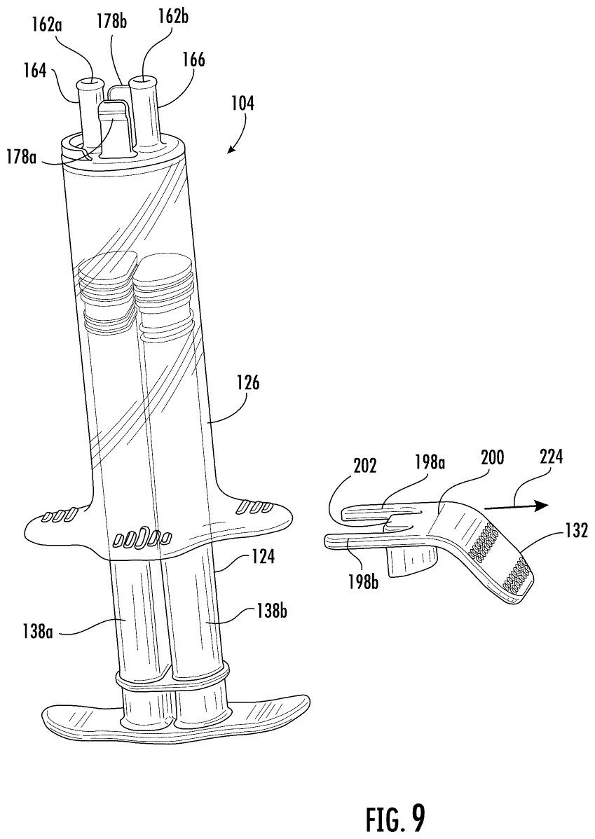

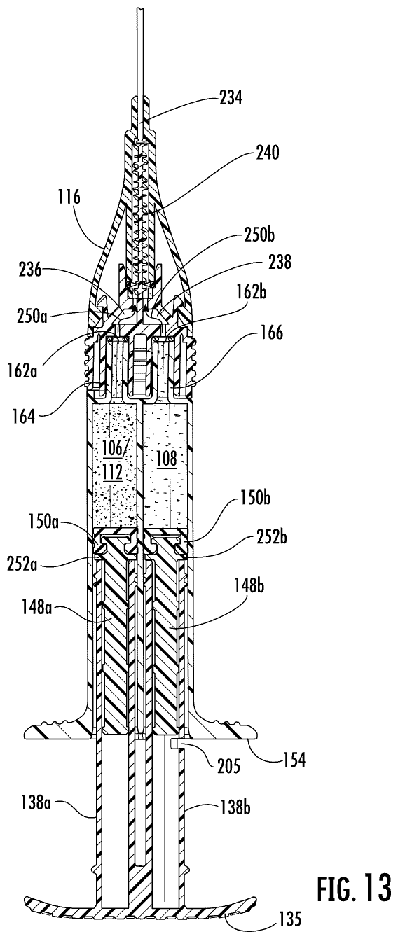

The retainer 132 may be removably positioned proximal to the proximal end 154 of the barrel portion 126. In some cases, the retainer 132 may form a snap fit, or other coupling mechanism with the plunger assembly 124. For example, the retainer 132 may include a pair of arms 198a, 198b (see, for example, FIG. 9) extending from a curved bridge region 200. The arms 198a, 198b may be configured to be positioned on opposing sides of the first and/or second plunger 138a, 138b while a tab 202 extending from the curved bridge region 200 is received within a groove or recess 205 (see, for example, FIG. 13) formed in the second plunger 138b. In other embodiments, the retainer 132 may form a friction fit with the plunger assembly 124. While the retainer 132 is positioned between the plunger assembly 124 and the barrel portion 126, distal actuation of the plunger assembly 124 and/or proximal actuation of the barrel portion 126 may be precluded or limited.

Generally, the plunger assembly 124 and the barrel portion 126 may be assembled in a telescoping arrangement. For example, a portion of plunger assembly 124 may be disposed within a portion of the barrel portion 126. More particularly, the plunger assembly 124 may be assembled with the barrel portion 126 such that the first plunger 138a and the first floating plunger 148a of the plunger assembly 124 are slidably disposed within the first reservoir 158a of the barrel portion 126 and the second plunger 138b and the second floating plunger 148b of the plunger assembly 124 are slidably disposed within the second reservoir 158b of the barrel portion 126. The plunger assembly 124 is movable, such as axially and/or longitudinally slidable, with respect to the barrel portion 126 to move (e.g., eject) materials out of and/or to move (e.g., aspirate) materials into first and/or second reservoirs 158a, 158b within the barrel portion 126. Similarly, the first floating plunger 148a may be axially and/or longitudinally slidable with respect to the first plunger 138a and the first reservoir 158a of the barrel portion 126 to allow materials out of and/or to move (e.g., aspirate) materials into first reservoir 158a within the barrel portion 126 and the second floating plunger 148b may be axially and/or longitudinally slidable with respect to the second plunger 138b and the second reservoir 158b of the barrel portion 126 to allow materials out of and/or to move (e.g., aspirate) materials into second reservoir 158b within the barrel portion 126.

The barrel portion 126 may be pre-loaded with some of the constituents required to form the injectable material. For example, a first constituent, such as, but not limited to, a first cross-linkable constituent 106 may be disposed within the first reservoir 158a of the barrel portion 126. In some examples, the first cross-linkable constituent 106 may be provided as a powder. A second constituent 108, such as, but not limited to, an accelerator, may be disposed within the second reservoir 158b of the barrel portion 126. In some examples, the second constituent 108 may be provided as a liquid. A third constituent 112, such as, but not limited to, a second cross-linkable constituent, may be disposed within the lumen 210 of the syringe 110. In some examples, the third constituent 112 may be provided as a liquid. The third constituent 112 may be fluidly isolated from the first constituent 106 until mixing is desired. Further, the second constituent 108 may be fluidly isolated from each of the first and third constituents 106, 112 until mixing is desired.

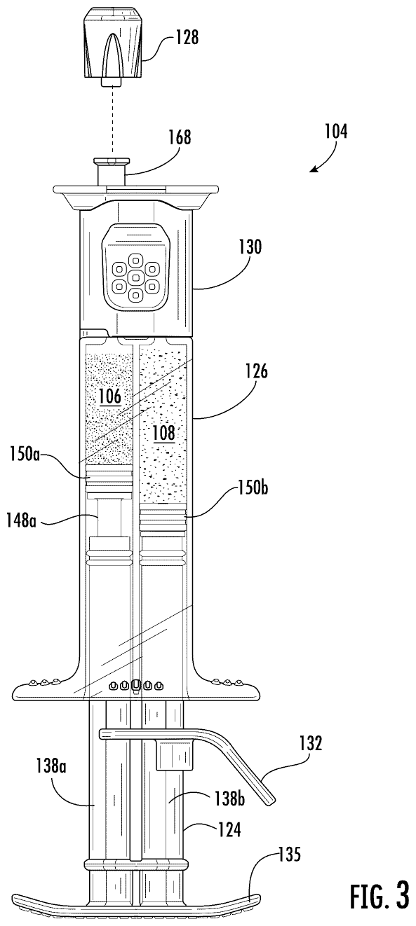

A method for dispensing or injecting the injectable material, along with additional features of the combining and/or delivery system 100 will now be described with respect to FIGS. 3-15. While certain steps are shown as a sequence between each figure, in other embodiments fewer steps are contemplated and the order by which steps are performed can be different than what is illustrated. To begin, the plug 128 is removed from the cap 130, as shown in FIG. 3, which depicts a side view of the illustrative multi-reservoir system 104 with the plug 128 removed. The plug 128 may be rotationally and/or axially displaced to uncouple the plug 128 from the cap 130. However, other movements or actions may be used to uncouple the plug 128 from the cap 130 from the barrel portion 126.

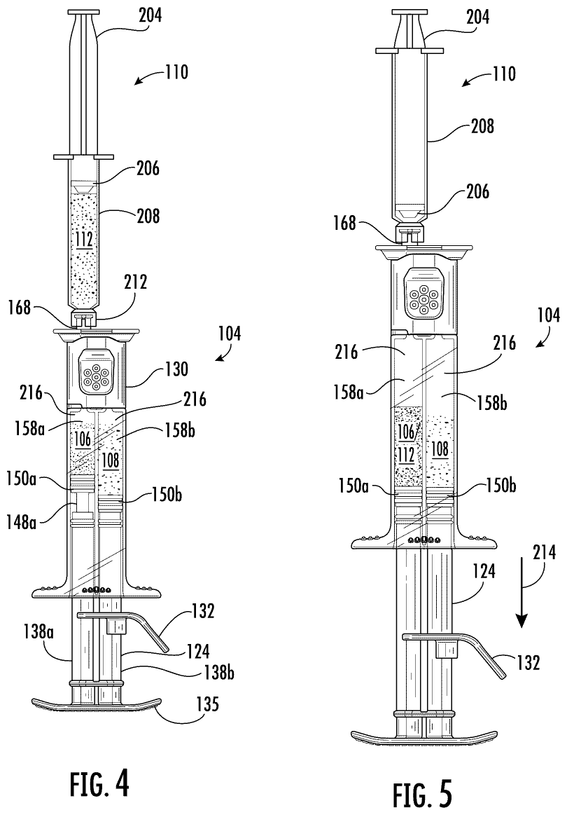

Once the plug 128 has been removed, the cap of the syringe 110, if so provided, may be also removed. In some cases, the cap of the syringe 110 may be removed prior to removing the plug 128 of the multi-reservoir system 104. With the plug 128 and the cap of the syringe 110 removed, the Luer lock 212 of the syringe 110 may be coupled with the port 168 of the cap 130, as shown in FIG. 4 which depicts a side view of the multi-reservoir system 104 coupled with the syringe 110. Connecting the syringe 110 with the port 168 of the cap 130 may fluidly couple the lumen 210 of the syringe 110 with the lumen 176 of the cap 130, first distal opening 162a, and the first reservoir 158a. The plunger 204 of the syringe 110 may then be axially displaced, as shown in FIG. 5 which depicts a side view of the multi-reservoir system 104 coupled with the syringe 110 in a dispensed configuration. As the plunger 204 of the syringe 110 is axially displaced towards the barrel portion 126, the third constituent 112 is dispensed from the lumen 210 of the syringe 110 into the first reservoir 158a of the barrel portion 126 via the port 168 of the cap 130, the cap lumen 176, and the first port 164. As the first floating plunger 148a floats within the first reservoir 158a of the barrel portion 126 and within the cavity 140a of the first plunger 138a, the first floating plunger 148a and the seal 150a may be axially displaced towards the proximal end 134 of the plunger assembly 124. Axially displacement of the first floating plunger 148a and the seal 150a may allow the volume of the first reservoir 158a to increase to accommodate the additional volume received from the syringe 110. In some examples, the pressure generated by the injection of the third constituent 112 into the first reservoir 158a may proximally displace the first floating plunger 148a which in turn may proximally displace the plunger assembly 124, as shown at arrow 214. It is contemplated that the pressure generated by injection of the third constituent 112 into the first reservoir 158a may not be sufficient to disengage the plunger assembly 124 from the barrel portion 126. Some air 216 may remain in the first reservoir 158a of the barrel portion 126 and/or in the second reservoir 158b of the barrel portion 126 after injection of the third constituent 112 into the first reservoir 158a.

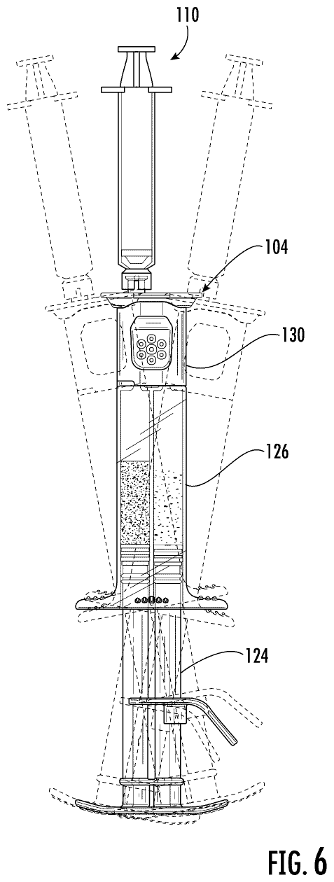

Once the third constituent 112 has been injected from the syringe 110 into the first reservoir 158a, the multi-reservoir system 104 may be shaken, as shown in FIG. 6, which depicts a side view of the illustrative multi-reservoir system 104 and syringe 110 being shaken, to mix the first constituent 106 and the third constituent 112 to form the precursor fluid 106/112. In some cases, the syringe 110 may remain coupled to the port 168 of the cap 130 as the multi-reservoir system 104 is shaken to prevent fluid from unintentionally spilling from the first distal opening 162a. In some cases, the syringe 110 may be removed and the plug 128 replaced to limit or prevent spilling. As used herein, the term “fluid” as it relates to constituents 106, 108, 112 of the system 100 is defined broadly and can include liquids, gels, oils and particulate matter such as granules, pellets, or powders, or any combination of liquids, gels, oils, and/or particulate matter (e.g., granules, pellets, or powders). As described above, in some examples, the third constituent 112 can be a diluent fluid solution and the first constituent 106 can include a cross-linkable polymer, such as PEG having a plurality of succinimidyl termini or any other agent mixable with diluent 112 to form precursor. The diluent 112 can include a low molecular weight compound that contains multiple nucleophilic groups, such as trilysine which contains multiple amino groups, dissolved in a low pH (4.0) aqueous solution, though other diluent fluid solutions are contemplated within the scope of this disclosure. Once mixed together, the precursor solution 106/112 can be formed in the first reservoir 158a.

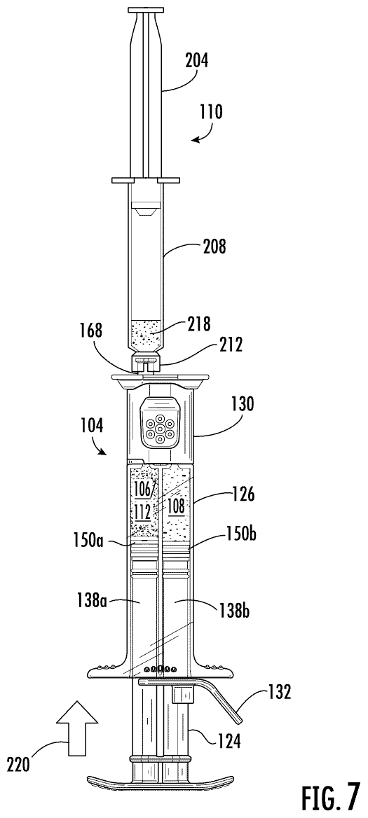

Next, the plunger assembly 124 may be actuated or distally advanced, as shown at arrow 220. In some cases, the plunger assembly 124 may be actuated or distally advanced while the syringe 110 remains coupled to the port 168 of the cap. For example, the plunger assembly 124 may be actuated or depressed axially towards the distal end 156 of the barrel portion 126 to remove air and/or to excess fluid from the first and/or second reservoirs 158a, 158b. This may be done with the cap 130 coupled to the distal end region of the barrel portion 126. The air and/or excess fluid 218 from the first reservoir 158a may pass through the distal opening 162a, into the lumen 176 of the cap 130, and finally into the lumen 210 of the syringe 110. The air and/or excess fluid from the second reservoir 158b may pass through the distal opening 162b and may be trapped within cavity 194 in the cap 130. The plunger assembly 124 may be actuated or distally advanced until the retainer 132 contacts the proximal end 154 of the barrel portion 126, as shown in FIG. 7, which depicts a side view of the illustrative multi-reservoir system 104 with the air/excess fluid purged. It is contemplated that the retainer 132 may be positioned at an axial location that ensures a desired amount of the precursor 106/112 and the second constituent 108 remain in the first and second reservoirs 158a, 158b, respectively.

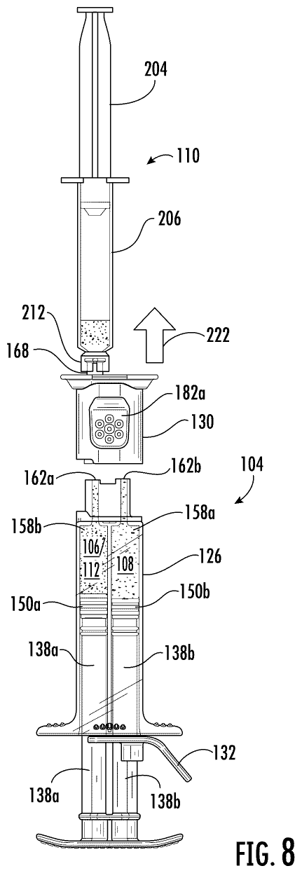

Next, the cap 130 and the syringe 110 may be removed as shown in FIG. 8 which depicts a side view of the illustrative multi-reservoir system 104 with the cap 130 and syringe 110 removed. In some examples, the cap 130 and/or syringe 110 may be axially displaced, as shown at arrow 222. However, this is not required. In some examples, the cap 130 and/or syringe 110 may be removed with other movements and/or forces. In some embodiments, the one or more buttons 182a, 182b may be depressed to release the latches 178a, 178b of the barrel portion 126 to allow the cap 130 to be removed from the barrel portion 126. It is further contemplated that the cap 130 and the syringe 110 may be removed substantially simultaneously. In other examples, the syringe 110 may be removed from the cap 130 and then the cap 130 removed from the barrel portion 126.

Next, the retainer 132 may be removed from the plunger assembly 124, as shown in FIG. 9, which depicts a perspective view of the illustrative multi-reservoir system 104 with the retainer 132 removed. The retainer 132 may be laterally displaced, as shown at arrow 224 to uncouple the retainer 132 from the plunger assembly 124. However, other movements or actions may be used to uncouple the retainer 132 from the plunger assembly 124. It is contemplated that the retainer 132 may be removed before or after hydrodissection, as desired.

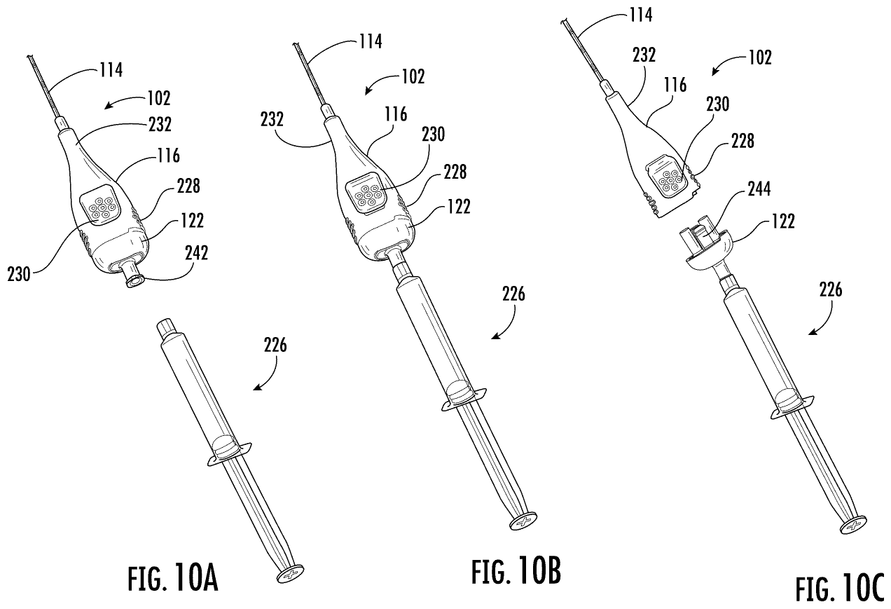

The multi-reservoir system 104 may be maintained in an upright orientation as hydrodissection is performed. Hydrodissection may be optionally performed before injecting the injectable material into the body. FIGS. 10A-10C depict an illustrative method for assembling and disassembling a syringe 226, such as, but not limited to, a saline syringe, for use in hydrodissection, with the injection system 102. Generally, the injection system 102 may be connected to the saline syringe 226. The needle 114 may then be positioned at the treatment site and saline injected to perform hydrodissection. In some examples, the saline syringe 226 may be provided separately from the combining and/or delivery system 100, although this is not required. Once the hydrodissection is complete, the saline syringe 226 may be uncoupled from the injection system 102 with the needle 114 remaining in position and primed at the treatment site (e.g., the distal end of the needle 114 remains in the body). However, this is not required. In some cases, the needle 114 may be removed from the body after hydrodissection.

Referring first to FIG. 10A, which depicts an exploded perspective view of the illustrative injection system 102 and saline syringe 226, the needle hub 116 may include a lower housing 228 configured to be gripped and squeezed by a user. One or more externally positioned buttons 230 can be positioned on an outer surface of lower housing 228. In some cases, two buttons 230 may be positioned on opposing sides of the lower housing 228. The button(s) 230 can be configured so that an actuating squeeze or other movement by a user causes latches of an adaptor or connector 122 and/or the barrel portion 126 of the multi-reservoir system 104 to release from mating apertures in the needle hub 116. However, other coupling mechanisms between the needle hub 116 and the connector 122 or barrel portion 126 are contemplated as needed or required. For example, and without limitation, snap fit connectors, magnetic connectors, female-male connectors, hook and loop fasteners, and the like are contemplated.

The needle hub 116 may also include a transitional portion 232 through which the needle 114 can be inserted. The transitional portion 232 can include a diameter (or cross-sectional dimension) smaller than the lower housing 228. In some examples, the transitional portion 232 can be tapered and/or include a textured outer surface. A central tubular lumen 234 connected to a first lumen 236 and a second lumen 238 (see, for example, FIG. 13) can pass through the needle hub 116 and be in fluid communication with the needle 114, when the needle 114 is connected to needle hub 116. The first and second lumens 236, 238 may be configured to be in fluid communication with one or more lumens of the connector 122 when connected thereto and to be in fluid communication with the distal openings 162a, 162b of the barrel portion 126 of the multi-reservoir system 104 when connected thereto. The connector 122 may be an adaptor, manifold, etc. configured to fluidly couple the single outlet of the saline syringe 226 to the first and second lumens 236, 238 of the needle hub 116. For example, the connector 122 may have a single fluid inlet for coupling to the outlet of the saline syringe 226 and two or more fluid outlets for coupling to first and second lumens 236, 238 of the needle hub 116. The connector 122 may include more than one fluid inlet or fewer than two or more than two fluid outlets as needed or desired. The first and second lumens 236, 238 of the needle hub 116 may intersect or merge at a mixing region 240 (see, for example, FIG. 13). The mixing region 240 may include a static mixer (not explicitly shown) configured to mix the fluids from the first and second lumens 236, 238.

To perform the hydrodissection, the injection system 102 and a saline syringe 226 may be required. While the syringe 226 is described as a saline syringe, the syringe 226 may include other fluids, as desired. To connect the injection system 102 with the saline syringe 226, a proximal end 242 of the connector 122, having a single fluid opening, may be aligned with the outlet of the saline syringe 226. The connector 122 may then be connected to the outlet of the saline syringe 226, as shown in FIG. 10B, which depicts a perspective view of the assembled illustrative injection system 102 and saline syringe 226. In some cases, the proximal end 242 of the connector 122 and the outlet of the saline syringe 226 may have mating Luer fittings. However, other connection mechanisms may be used as desired, such as, but not limited to, friction fits, snap fits, threaded engagements, etc. Hydrodissection may then be performed. Once the hydrodissection is complete, the saline syringe 226 may be uncoupled from the injection system 102 while leaving the needle 114 at the treatment site. It is further contemplated that the connector 122 may also be uncoupled from the injection system 102. In some cases, the connector 122 and the saline syringe 226 may be uncoupled from the injection system 102 substantially simultaneously. For example, actuation of the button 230 may cause one or more latches 244 of the connector 122 to release from a mating recess of the needle hub 116. In some cases, a latch 244 may be provided on each of the opposing sides of the connector 122, although this is not required. Proximal retraction of the saline syringe 226 while the button 230 of the needle hub 116 is actuated may release both the connector 122 and the saline syringe 226 from the needle hub 116, as shown in FIG. 10C, which depicts a perspective view of the unassembled illustrative injection system 102 and saline syringe 226. The connector 122 may remain coupled to the saline syringe 226 or may be subsequently uncoupled from the saline syringe 226, if so desired. While the connector 122 and the saline syringe 226 have been described as being uncoupled substantially simultaneously, in some cases, the saline syringe 226 may first be uncoupled from the connector 122 and then the connector 122 uncoupled from the needle hub 116.

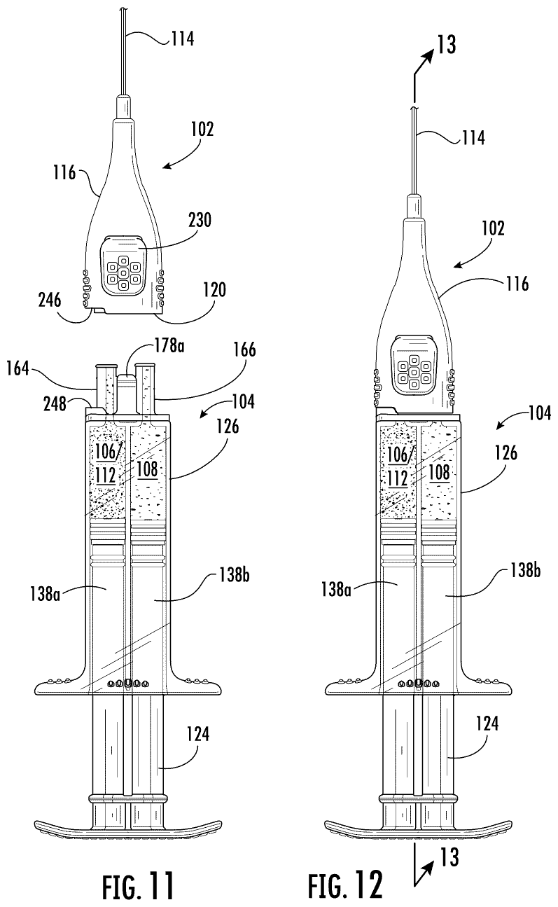

Next, the multi-reservoir system 104 may be connected to the needle hub 116. To do so, the first and second lumens 236, 238 of the needle hub 116 may be aligned with the distal openings 162a, 162b of the barrel portion 126, as shown in FIG. 11 which depicts a side view of the unassembled injection system 102 and multi-reservoir system 104. In some cases, a cut-out 246 on a proximal end 120 of the needle hub 116 may be aligned with a lip or ridge 248 extending distally from a distal end region of the barrel portion 126, although this is not required. When assembled, the lower housing 228 of the needle hub 116 may be slid over the first and second ports 164, 166 of the barrel portion 126, as shown in FIG. 12 which depicts a side view of the assembled injection system 102 and multi-reservoir system 104.

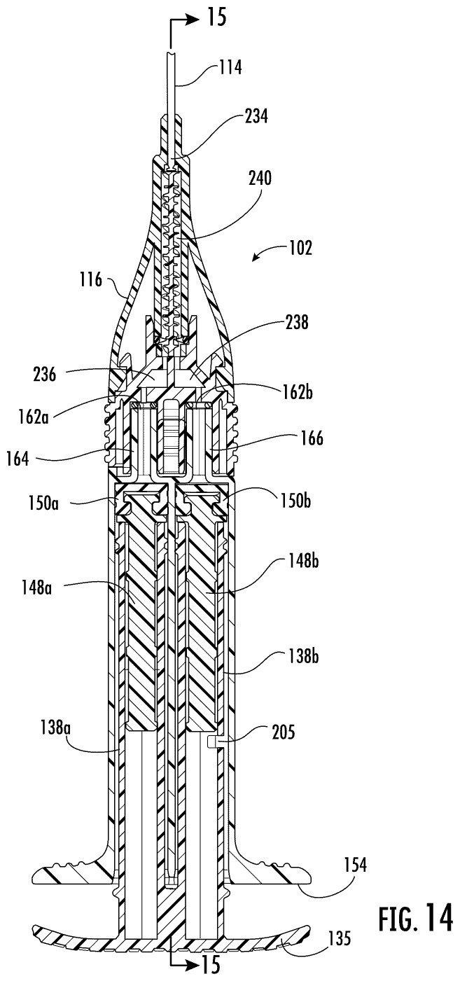

Referring additionally to FIG. 13, which depicts the cross-sectional view of the assembled injection system 102 and multi-reservoir system 104 taken at line 13-13 of FIG. 12 and FIG. 14, which depicts the cross-sectional view of FIG. 13 in a dispensed configuration, once the multi-reservoir system 104 has been assembled with the injection system 102, the plunger assembly 124 can be actuated or distally advanced to cause the precursor fluid 106/112 (e.g., resulting from the mixture of the first constituent 106 and the third constituent 112) disposed in the first reservoir 158a of the barrel portion 126 and the second constituent 108 (e.g., the accelerator, such as, but not limited to a basic buffer solution) disposed in the second reservoir 158b of the barrel portion 126 to be dispensed from the distal openings 162a, 162b. The first and second lumens 236, 238 of the needle hub 116 may be in fluid communication with the first and second reservoirs 158a, 158b and the distal openings 162a, 162b. As the plunger assembly 124 is actuated or distally advanced, fluid may exit the first and second reservoirs 158a, 158b of the barrel portion 126 and enter the first and second lumens 236, 238 of the needle hub 116 along flow paths 250a, 250b. For example, the distal end of the first plunger 138a may engage a radially extending flange 252a of the first floating plunger 148a to actuate or distally advance the first floating plunger 148a and seal 150a to expel fluid (precursor 106/112) from the first distal opening 162a. Similarly, the second plunger 138b may engage a radially extending flange 252b of the second floating plunger 148b to actuate or distally advance the second floating plunger 148b and seal 150b to expel fluid (second constituent 108) from the second distal opening 162b. The first and second lumens 236, 238 of the needle hub 116 may be connected with a mixing region 240. The mixing region 240 may be configured to mix or combine the precursor 106/112 and the second constituent 108 prior to the constituents exiting the needle 114. For example, the mixing region 240 may mix or combine the precursor 106/112 and the second constituent 108 to form the injectable composition before the resulting mixture enters the central lumen 234 of the needle hub 116 which in turn is in fluid communication with a lumen of the needle 114. The injectable composition may continue egressing through the needle 114 and ultimately to the treatment site. The injectable composition formed from the combination of the precursor 106/112 and the second constituent 108 may attain its final desired properties and/or reach its final form in situ or at the target site. In the fully dispensed configuration, as shown in FIG. 14, the seals 150a, 150b of the first and second plungers 138a, 138b may contact a distal wall of the barrel portion 126, although this is not required.

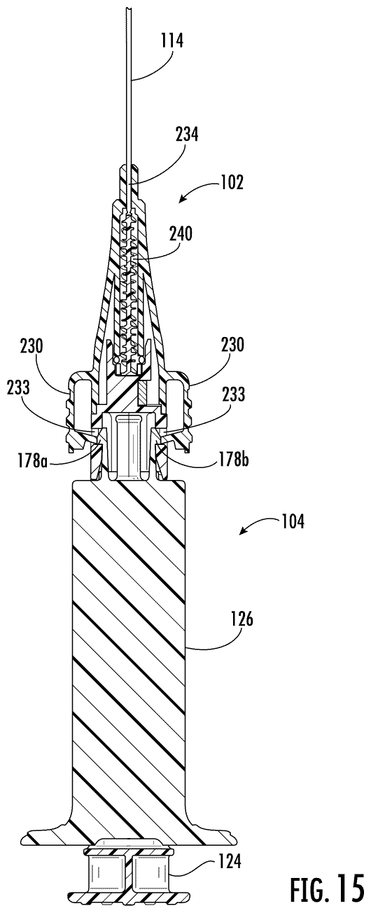

FIG. 15, which depicts an alternative cross-sectional view of the assembled injection system 102 and multi-reservoir system 104 in a dispensed configuration taken at line 15-15 of FIG. 14, when the needle hub 116 is assembled with the distal end region of the barrel portion 126, one or more tabs 178a, 178b of the barrel portion 126 may be received within one or more mating apertures 233 of the needle hub 116. The one or more apertures 233 may be disposed below the one or more buttons 230 such that actuating (e.g., depressing) the one or more buttons 230 is configured to disengage the one or more tabs 178a, 178b of the barrel portion 126 from the one or more mating apertures 233 to allow the injection system 102 to be uncoupled from the multi-reservoir system 104.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A system for producing a mixture to deliver to a treatment site, comprising:
a needle hub;
a multi-reservoir system, the multi-reservoir system comprising:
a plunger assembly including a first tubular plunger and a second tubular plunger;
a barrel portion including a housing defining a first reservoir in fluid communication with a first port and configured to contain a first constituent and a second reservoir in fluid communication with a second port and configured to contain a second constituent; and
a first floating plunger movably disposed relative to the plunger assembly and at least partially within the first reservoir; and
a syringe defining a lumen configured to contain a third constituent;
wherein a distal end of the syringe is configured to be releasably coupled to the barrel portion such that actuation of the syringe causes the third constituent to be injected into the first reservoir via the lumen of the cap to mix with the first constituent and form a precursor; and
wherein actuating the plunger assembly relative to the barrel portion causes the precursor and the second constituent to be delivered into the needle hub.

2. The system of claim 1, further comprising a cap removably coupled to a distal portion of the barrel portion, the cap including a port and a lumen extending from the port to a proximal end of the cap, wherein the distal end of the syringe is configured to be releasably coupled to the port of the cap.

3. The system of claim 1, further comprising a removable retainer positioned between a proximal end of the barrel portion and a proximal end of the plunger assembly, the removable retainer limiting movement of the plunger assembly relative to the barrel portion.

4. The system of claim 2, further comprising a plug removably coupled with the port of the cap.

5. The system of claim 2, wherein the cap further comprises a cavity in selective fluid communication with the second reservoir.

6. The system of claim 5, further comprising a floating seal disposed within the cavity of the cap, the floating seal configured to selectively seal the second port.

7. The system of claim 1, wherein the first floating plunger is unsecured to the plunger assembly or the barrel portion.

8. The system of claim 1, further comprising a second floating plunger movably disposed relative to the plunger assembly and at least partially within the second reservoir.

9. The system of claim 8, wherein the second floating plunger is unsecured to the plunger assembly or the barrel portion.

10. The system of claim 1, wherein the needle hub comprises a first lumen in fluid communication with the first reservoir of the barrel portion via the first port, a second lumen in fluid communication with the second reservoir of the barrel portion via the second port, a central lumen configured to be in fluid communication with a needle, and a mixing region connecting the first and second lumens with the central lumen.

11. The system of claim 1, wherein the needle hub is removably coupled to a distal end region of the barrel portion of the multi-reservoir system.

12. The system of claim 1, wherein the plunger assembly and the barrel portion are assembled in a telescoping arrangement.

13. A kit for producing a mixture for delivery to a treatment site, the kit comprising:
a multi-reservoir system, the multi-reservoir system comprising:
a plunger assembly including a first tubular plunger and a second tubular plunger;
a barrel portion including a housing defining a first reservoir in fluid communication with a first port and configured to contain a first constituent and a second reservoir in fluid communication with a second port and configured to contain a second constituent;
a first floating plunger movably disposed relative to the plunger assembly and at least partially within the first reservoir; and
a cap removably coupled to a distal portion of the barrel portion, the cap including a port and a lumen extending from the port to a proximal end of the cap;
a syringe defining a lumen configured to contain a third constituent; and
an injection system, the injection system comprising:
a needle hub; and
a needle coupled to the needle hub.

14. The kit of claim 13, further comprising a removable retainer positioned between a proximal end of the barrel portion and a proximal end of the plunger assembly, the removable retainer configured to limit movement of the plunger assembly relative to the barrel portion.

15. The kit of claim 13, further comprising a plug removably coupled with the port of the cap.

16. The kit of claim 13, further comprising a second floating plunger movably disposed relative to the plunger assembly and at least partially within the second reservoir.

17. The kit of claim 13, further comprising a connector, the connector configured to couple the needle hub to an additional syringe.

18. A method for producing a mixture with a mixing system to deliver to a treatment site, the method comprising:
coupling a distal end of a syringe with a first port of the multi-reservoir system;
injecting a first constituent into a first reservoir of the multi-reservoir system to mix the first constituent with a second constituent contained in the first reservoir of the multi-reservoir system and form a precursor;
coupling a needle hub having a mixing region to a distal end of the multi-reservoir system; and
actuating a plunger assembly to distally advance the precursor and a third constituent disposed within a second reservoir of the multi-reservoir system into the mixing region of the needle hub to form an injectable mixture.

19. The method of claim 18, further comprising removing a retainer from the plunger assembly prior to actuating the plunger assembly.

20. The method of claim 18, further comprising actuating the plunger assembly after removing a plug from a port of a cap of the multi-reservoir system and prior to removing the cap to purge air from the multi-reservoir system.
